(19) United States
(12) Patent Application Publication
(10) Pub. No.: US 2026/0042727 A1
(43) Pub. Date: Feb. 12, 2026

(54) COMPLEX OF 3-HYDROXY-3-METHYLBUTYRIC ACID AND SALT THEREOF, AND PREPARATION METHOD THEREFOR

(71) Applicant: NANJING NUTRABUILDING BIO-TECH CO., LTD., Nanjing (CN)

(72) Inventors: Liang RONG, Nanjing (CN); Youjian CHEN, Nanjing (CN); Xi ZHU, Nanjing (CN); Shawn WELLS, Nanjing (CN); Kylin LIAO, Nanjing (CN)

(21) Appl. No.: 19/273,427

(22) Filed: Jul. 18, 2025

Related U.S. Application Data
(63) Continuation of application No. PCT/CN2024/073297, filed on Jan. 19, 2024.

(30) Foreign Application Priority Data
Jan. 19, 2023 (WO) PCT/CN2023/073064

(51) Int. Cl. C07C 59/01; C07C 51/41

(52) U.S. Cl. CPC C07C 59/01; C07C 51/41; C07B 2200/13

(57) ABSTRACT

Disclosed are a complex of 3-hydroxy-3-methylbutyric acid and salt thereof as well as preparation method thereof. The complex effectively solves the problems of low solubility, poor stability, acid/salt load, excessive calcium intake and gastrointestinal side effects associated with existing products, has good taste comfort, and improves flavor. In addition, it has the advantages of better increasing muscle weight, improving muscle performance, joint function, body composition or enhancing health. It can be better used as a dietary or nutritional supplement.

### CROSS REFERENCE TO RELATED APPLICATION

This application is a continuation application of International Patent Application No. PCT/CN2024/073297, filed on Jan. 19, 2024, which claims the priority to International Patent Application No. PCT/CN2023/073064, filed on Jan. 19, 2023. The contents of International Patent Application No. PCT/CN2024/073297 and International Patent Application No. PCT/CN2023/073064 are incorporated herein by reference in their entireties.

### TECHNICAL FIELD

The present disclosure belongs to the technical field of dietary or nutritional supplements, and specifically relates to a complex of 3-hydroxy-3-methylbutyric acid and salt thereof as well as preparation method thereof.

### BACKGROUND

At present, people's demand for dietary or nutritional supplements is increasing, aiming at improving individual health and reducing disease risk.

A large number of studies on 3-hydroxy-3-methylbutyric acid (HMB) show that intake of HMB can improve muscle performance: increase muscle strength, promote muscle recovery, reduce muscle atrophy, reduce muscle injury, maintain myoglobin level, improve muscle aerobic capacity, reduce or prevent the loss of contractile substances in muscle, and increase the amount of hemoglobin in blood. Improve body composition: reduce body fat, increase fat-free mass, increase or maintain fat-free mass, and increase lean tissue development or lean body mass; enhance or promote lipolysis, reduce fat, increase fat oxidation of adipocytes, induce biosynthesis of adipocytes and muscle mitochondria, increase energy consumption and reduce overall weight. Improve joint function and health: increase joint stability and reduce joint stiffness. Promote or improve the recovery and healing of soft tissue injury. Improve and enhance health in many ways.

Intake of HMB can also improve the perception of emotional state and enhance or improve cognitive ability or function. Reduce insulin resistance and improve glucose tolerance. Prevent or alleviate diarrhea or intestinal inflammatory diseases, chronic inflammatory diseases and accidental weight loss. Regulate autophagy and lipophagy; Treat, prevent, inhibit, slow down or reduce autophagy-mediated disorders or diseases, including cardiovascular diseases, type II diabetes, neurodegenerative diseases, metabolic syndrome, liver diseases, decreased metabolic rate, muscle diseases, osteoarthritis, sarcopenia, cancer, obesity and insulin resistance; Treat, inhibit, reduce or slow down age-related diseases in mammals, including obesity, sarcopenia and/or sarcopenia obesity. Inhibit or relieve depression, asthma, allergy and cancer. Promote nitrogen retention and improve nutrition in protein. Reduce or eliminate microbial pollution in animal feed.

Commercially, HMB only circulates in the market in any form of free acid or calcium salt. Especially as a supplement and health food, calcium salt is basically used because it is a powder with excellent operability. However, the existing known products have the disadvantages of limiting their use. In the field of preparations, there are some problems that calcium from calcium salts can easily combine with other components such as phosphoric acid to form insoluble salts, and high-concentration solutions cannot be prepared. It has been reported that excessive intake of calcium will increase the risk of death caused by cardiovascular disease and ischemic heart disease. However, HMB in the form of free acid has some disadvantages.

Compared with the existing HMB free acid or calcium salt products, the complex of the present disclosure can effectively avoid or balance the above problems, so as to be better added or used as a dietary or nutritional supplement. The complex of HMB acid and salt of the present disclosure not only effectively improves the utilization of HMB, accelerates the absorption of HMB, but also shows high solubility, does not form insoluble salt or induce electrolyte abnormality, and has many advantages. In addition, the complex of HMB acid and salt has good taste comfort and excellent flavor improvement effect.

### SUMMARY

In one aspect, the present disclosure provides a 3-hydroxy-3-methylbutyric acid·3-hydroxy-3-methylbutyrate complex and composition thereof, where a salt in the 3-hydroxy-3-methylbutyric acid·3-hydroxy-3-methylbutyrate complex is a sodium salt, potassium salt, calcium salt, magnesium salt, ammonium salt, amino acid salt.

In some embodiments, the complex is in a solid form.

In some embodiments, the complex is in crystalline form.

In some embodiments, the sodium salt complex has the following structure:

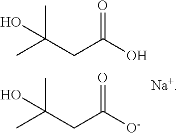

In some embodiments, the X-ray powder diffraction pattern of the sodium salt complex includes peaks at diffraction angles (2θ) of 8.8±0.2°, 18.1±0.2°, 24.3±0.2°, and 35.6±0.2°.

In some embodiments, the X-ray powder diffraction pattern of the sodium salt complex further includes one or more peaks at diffraction angles (2θ) of 7.9±0.2°, 19.0±0.2°, 28.6±0.2°.

In some embodiments, the X-ray powder diffraction pattern of the sodium salt complex further includes one or more peaks at diffraction angles (2θ) of 17.6±0.2°, 21.4±0.2°, 36.7±0.2°.

In some embodiments, the X-ray powder diffraction pattern of the sodium salt complex is shown in FIG. 1.

In some embodiments, the potassium salt complex has the following structure:

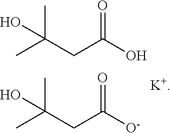

In some embodiments, the X-ray powder diffraction pattern of the potassium salt complex includes peaks at diffraction angles (2θ) of 15.9±0.2°, 18.3±0.2°, 20.6±0.2°, and 34.7±0.2°.

In some embodiments, the X-ray powder diffraction pattern of the potassium salt complex further includes one or more peaks at diffraction angles (2θ) of 10.6±0.2°, 17.9±0.2°, 22.6±0.2°.

In some embodiments, the X-ray powder diffraction pattern of the potassium salt complex further includes one or more peaks at diffraction angles (2θ) of 11.9±0.2°, 18.5±0.2°, 30.7±0.2°.

In some embodiments, the X-ray powder diffraction pattern of the potassium salt complex is shown in FIG. 2.

In some embodiments, the calcium salt complex has the following structure:

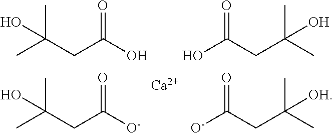

In some embodiments, the X-ray powder diffraction pattern of the calcium salt complex includes peaks at diffraction angles (2θ) of 9.0±0.2°, 18.8±0.2°, 26.4±0.2°, and 30.3±0.2°.

In some embodiments, the X-ray powder diffraction pattern of the calcium salt complex further includes one or more peaks at diffraction angles (2θ) of 8.1±0.2°, 13.1±0.2°, 20.4±0.2°.

In some embodiments, the X-ray powder diffraction pattern of the calcium salt complex further includes one or more peaks at diffraction angles (2θ) of 16.5±0.2°, 25.4±0.2°, 34.2±0.2°.

In some embodiments, the X-ray powder diffraction pattern of the calcium salt complex is shown in FIG. 3.

In some embodiments, the magnesium salt complex has the following structure:

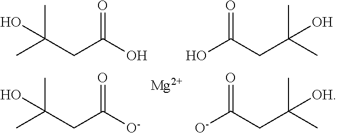

In some embodiments, the X-ray powder diffraction pattern of the magnesium salt complex includes peaks at diffraction angles (2θ) of 8.9±0.2°, 16.5±0.2°, 25.3±0.2°, and 37.4±0.2°.

In some embodiments, the X-ray powder diffraction pattern of the magnesium salt complex further includes one or more peaks at diffraction angles (2θ) of 9.4±0.2°, 18.8±0.2°, 30.2±0.2°.

In some embodiments, the X-ray powder diffraction pattern of the magnesium salt complex further includes one or more peaks at diffraction angles (2θ) of 13.0±0.2°, 20.3±0.2°, 34.1±0.2°.

In some embodiments, the X-ray powder diffraction pattern of the magnesium salt complex is shown in FIG. 4.

In some embodiments, the complex is prepared as food, beverage, supplement, nutritional product or pharmaceutical preparation.

In another aspect, the present disclosure provides a 3-hydroxy-3-methylbutyric acid·3-hydroxy-3-methylbutyrate complex, where the complex is obtained by the following method:
(1) a substance B is obtained by one of the following methods: mixing 3-hydroxy-3-methylbutyric acid and 3-hydroxy-3-methylbutyrate salt; or adding 3-hydroxy-3-methylbutyric acid into an aqueous solution of an alkaline compound, stirring, removing water; or reacting alkyl 3-hydroxy-3-methylbutyrate with water under heating in the presence of a catalyst, cooling and filtering, adding an aqueous solution of an alkaline compound to the filtrate, distilling under reduced pressure to remove water;
(2) adding one or more solvents selected from the following into the substance B obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

In some embodiments, the complex is in crystalline form.

In another aspect, the present disclosure provides a method for preparing the complex, including the following steps:
(1) a substance B is obtained by one of the following methods: mixing 3-hydroxy-3-methylbutyric acid and 3-hydroxy-3-methylbutyrate salt; or adding 3-hydroxy-3-methylbutyric acid into an aqueous solution of an alkaline compound, stirring, removing water; or reacting alkyl 3-hydroxy-3-methylbutyrate with water under heating in the presence of a catalyst, cooling and filtering, adding an aqueous solution of an alkaline compound to the filtrate, distilling under reduced pressure to remove water;
(2) adding one or more solvents selected from the following into the substance B obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

In some embodiments, in step (2), the alcohol is methanol, ethanol, isopropanol, n-butanol; the halogenated hydrocarbon is chlorobenzene, dichlorobenzene, dichloromethane; the ketone is acetone, methyl butanone, methyl isobutyl ketone; the ester is ethyl acetate, isopropyl acetate, n-butyl acetate, isobutyl acetate.

In some embodiments, in step (1), the alkaline compound is hydroxide, carbonate, bicarbonate, methoxide, acetate or formate of sodium, potassium, calcium or magnesium, ammonia water, triethylamine, N,N-diisopropylethylamine; the alkyl 3-hydroxy-3-methylbutyrate is methyl 3-hydroxy-3-methylbutyrate, ethyl 3-hydroxy-3-methylbutyrate, propyl 3-hydroxy-3-methylbutyrate, isopropyl 3-hydroxy-3-methylbutyrate, butyl 3-hydroxy-3-methylbutyrate, isobutyl 3-hydroxy-3-methylbutyrate.

In another aspect, the present disclosure provides a composition including an effective amount of the complex and a pharmaceutically acceptable carrier.

In some embodiments, the composition is used as a substance for improving muscle performance, joint function, body composition or enhancing health.

In some embodiments, the composition is prepared as food, beverage, supplement, nutritional product or pharmaceutical preparation.

In another aspect, the present disclosure provides use of the complex in preparing a substance for improving muscle performance, joint function, body composition or enhancing health.

In some embodiments, the substance is nutritional supplement, energy therapy, medical treatment or strength and/or endurance exercise supplement.

In another aspect, the present disclosure provides use of a composition in preparing a substance for improving muscle performance, joint function, body composition or enhancing health, and the composition includes the complex of the present disclosure and a pharmaceutically acceptable carrier.

In some embodiments, the substance is nutritional supplement, energy therapy, medical treatment or strength and/or endurance exercise supplement.

Compared with the prior art, the complex of the present disclosure has the beneficial effects that the complex of the present disclosure effectively avoids the instability caused by the use of HMB acid and the problems of excessive calcium and metal ions caused by the use of HMB calcium salt or other metal salts. When administered to a subject, the complex shows a better comprehensive effect than acid or salt alone or simply physically mixed components. On the one hand, the complex of the present disclosure solves the problems of acid instability and low operability, and at the same time solves the problem of electrolyte imbalance caused by high salt load of HMB calcium salt or other metal salts, and there is no uneven mixing of physical mixing, so it can have a wide application prospect in the field of dietary supplements or foods.

### BRIEF DESCRIPTION OF THE DRAWINGS

FIG. 1 shows an XRPD pattern of 3-hydroxy-3-methylbutyric acid·sodium 3-hydroxy-3-methylbutyrate complex of the present disclosure.

FIG. 2 shows an XRPD pattern of 3-hydroxy-3-methylbutyric acid·potassium 3-hydroxy-3-methylbutyrate complex of the present disclosure.

FIG. 3 shows an XRPD pattern of 3-hydroxy-3-methylbutyric acid·calcium 3-hydroxy-3-methylbutyrate complex of the present disclosure.

FIG. 4 shows an XRPD pattern of 3-hydroxy-3-methylbutyric acid·magnesium 3-hydroxy-3-methylbutyrate complex of the present disclosure.

FIG. 5 shows an infrared spectrogram (IR) of 3-hydroxy-3-methylbutyric acid·sodium 3-hydroxy-3-methylbutyrate complex of the present disclosure.

FIG. 6 shows an infrared spectrogram (IR) of 3-hydroxy-3-methylbutyric acid·potassium 3-hydroxy-3-methylbutyrate complex of the present disclosure.

FIG. 7 shows an infrared spectrogram (IR) of 3-hydroxy-3-methylbutyric acid·calcium 3-hydroxy-3-methylbutyrate complex of the present disclosure.

FIG. 8 shows an infrared spectrogram (IR) of 3-hydroxy-3-methylbutyric acid·magnesium 3-hydroxy-3-methylbutyrate complex of the present disclosure.

FIG. 9 shows a TGA diagram of 3-hydroxy-3-methylbutyric acid·sodium 3-hydroxy-3-methylbutyrate complex of the present disclosure.

FIG. 10 shows a TGA diagram of 3-hydroxy-3-methylbutyric acid·potassium 3-hydroxy-3-methylbutyrate complex of the present disclosure.

FIG. 11 shows a TGA diagram of 3-hydroxy-3-methylbutyric acid·calcium 3-hydroxy-3-methylbutyrate complex of the present disclosure.

FIG. 12 shows a TGA diagram of 3-hydroxy-3-methylbutyric acid·magnesium 3-hydroxy-3-methylbutyrate complex of the present disclosure.

FIG. 13 shows a Raman spectrogram of 3-hydroxy-3-methylbutyric acid·sodium 3-hydroxy-3-methylbutyrate complex of the present disclosure.

FIG. 14 shows a Raman spectrogram of 3-hydroxy-3-methylbutyric acid·potassium 3-hydroxy-3-methylbutyrate complex of the present disclosure.

FIG. 15 shows a DSC thermogram of the 3-hydroxy-3-methylbutyric acid·sodium 3-hydroxy-3-methylbutyrate complex of the present disclosure.

FIG. 16 shows a DSC thermogram of the 3-hydroxy-3-methylbutyric acid·potassium 3-hydroxy-3-methylbutyrate complex of the present disclosure.

FIG. 17 shows a DSC thermogram of the 3-hydroxy-3-methylbutyric acid·calcium 3-hydroxy-3-methylbutyrate complex of the present disclosure.

FIG. 18 shows a DSC thermogram of the 3-hydroxy-3-methylbutyric acid·magnesium 3-hydroxy-3-methylbutyrate complex of the present disclosure.

FIG. 19 shows the muscle weight results of experimental mice at 7 weeks.

### DETAILED DESCRIPTION OF THE EMBODIMENTS

Reference will now be made in detail to the preferred embodiments of the present disclosure, examples of which are further illustrated. While the present disclosure will be described in conjunction with the preferred embodiments, it will be understood that they are not intended to limit the present disclosure to these embodiments. On the contrary, the present disclosure is intended to cover alternatives, modifications, and equivalents, which may be included within the spirit and scope of the present disclosure as defined by the claims. Furthermore, in the detailed description of the present disclosure, numerous specific details are set forth in order to provide a thorough understanding of the present disclosure. However, it will be obvious to one of ordinary skill in the art that the present disclosure may be practiced without these specific details. In other instances, well known methods, procedures, components, and other features have not been described in detail as not to unnecessarily obscure aspects of the present disclosure.

The 3-hydroxy-3-methylbutyric acid·3-hydroxy-3-methylbutyrate complex of the present disclosure includes 3-hydroxy-3-methylbutyric acid and 3-hydroxy-3-methylbutyrate in any suitable ratio, and the complex can be hydrate with fixed water ratio, non-hydrate, and corresponding crystal forms. 3-hydroxy-3-methylbutyrate can be sodium salt, potassium salt, ammonium salt, amino acid salt, calcium salt and/or magnesium salt or other suitable salts. The amino acid salt is preferably a basic amino acid salt, especially arginine salt, lysine salt, histidine salt and ornithine salt.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As use herein, the terms “about” and “approximately” provide numerical flexibility by providing endpoints where a given value can be “slightly above” or “less than”. The flexibility of this term can be determined by specific variables and based on experience and related descriptions herein within the knowledge of those skilled in the art.

3-hydroxy-3-methylbutyric acid, also known as β-hydroxy-β-methylbutyric acid, β-hydroxyisovaleric acid, 3-hydroxyisovaleric acid or HMB, has the general formula C₅H₁₀O₃. HMB is a metabolite of leucine, which is an essential amino acid. It is a beneficial compound with the functions of reducing muscle tissue injury during exercise, increasing muscle and muscle strength, improving the recovery ability of muscle tissue injury, improving the balance of muscle synthesis and decomposition, maintaining muscle and muscle strength, reducing body fat, tightening and improving basal metabolism. “3-hydroxy-3-methylbutyric acid derivative” refers to a compound with the following chemical structure: where X is hydrogen, metal ion, ammonium, amino cation (such as amino acid), etc.

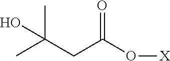

When X is a hydrogen, the compound is 3-hydroxy-3-methylbutyric acid. When X is a metal ion or an amino cation, the compound is 3-hydroxy-3-methylbutyrate salt. The foregoing compounds can be in any desired physical form, such as crystalline, powder, solid, liquid, solution, suspension, or gel.

As used herein, the term “administration” refers to the process of delivering a disclosed composition or active ingredient to a subject. The complexes of the present disclosure can be administered in a variety of suitable ways, including orally, intragastrically, and parenterally (e.g., intravenous and intraarterial as well as other suitable parenteral routes), and the like, so as to exert the desired effects. The complex of the present disclosure can be administered to a subject at a therapeutically effective dose and/or at an effective frequency. In some embodiments, a single dose will include an amount of about 0.1-30 grams, about 0.2-25 grams, about 0.5-20 grams, about 1-10 grams, or about 2-6 grams. In some embodiments, multiple doses of complex are administered over a period of time. The frequency of administration of the complex can vary depending on any of a variety of factors, such as timing of treatment from previous treatments, objectives of the treatment, and the like. The duration of administration of the complex (e.g., the period of time over which the agent is administered), can vary depending on any of a variety of factors, including subject response, desired effect of treatment, etc.

As used herein, the term “effective amount” refers to the amount required to achieve an effect as taught herein. The amount to be administered can vary according to factors such as the degree of susceptibility of the individual, the age, sex, and weight of the individual, idiosyncratic responses of the individual, and the like. In accordance with the present disclosure, a suitable single dose size is a dose that is capable of achieve the above effects when administered one or more times over a suitable time period.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

In some embodiments, the complex of the present disclosure can be prepared into a composition together with an alimentarily or pharmaceutically acceptable carrier. In the present disclosure, the application form of providing composition involves liquid or solid fillers, diluents, excipients, solvents or encapsulate materials. Each carrier must be “acceptable”, which means that it is compatible with other components of the composition and harmless to the subject, that is, suitable for consumption or nutritionally acceptable. The carriers include non-toxic and compatible substances commonly used in health foods, dietary supplements and pharmaceutical preparations, such as sugar, starch, cellulose and its derivatives, powdered tragacanth, malt, gelatin, talc, oil, diol, polyol, ester, agar, alginic acid, pyrogen free water, isotonic saline, etc.

In some embodiments, the complexes of the present disclosure can be administered together with other supplements, such as vitamins, minerals, nootropics, and others known in the art. Examples of vitamins, minerals and herbal supplements that can be added to the ketogenic compositions include one or more of vitamin A, vitamin C, vitamin D3, vitamin E, niacin, vitamin B6, folic acid, 5-MTHF, vitamin B12, iodine, zinc, copper, manganese, chromium, caffeine, theobromine, theacrine, methylliberine, huperzine A, epicatechins, and enzymes.

In some embodiments, the complexes of the present disclosure may be provided as a solid or powder form. The compositions in such solid form may be formulated so as to provide for sufficient ease of handling and manufacturability. The complexes may be provided as a liquid, such as in the form of a shot or mouth spray for fast delivery and absorption. Liquid forms may include one or more liquid carriers, such as water, ethanol, glycerin, propylene glycol, 1,3-propandiol, and the like.

In some embodiments, the complex of the present disclosure can be used as suppository, tablet, pill, granule, powder, film, capsule, beverage, aerosol, alcohol, tincture, tonic, liquid suspension or syrup.

The complex and/or composition of the present disclosure can be prepared as food and beverage products for human consumption, as well as nutritional supplements, energy therapy, medical treatment or strength and/or endurance exercise supplements etc., as a substance for increasing muscle weight and losing weight, improving muscle performance, joint function, body composition or enhancing health. The obtained product can show balance effects of reduced acidity, lower hygroscopicity, better taste, better palatability, uniform appearance, excellent stability and good solubility effect, and has no problems of intestinal side effects, electrolyte imbalance and high salt load.

The following examples are illustrative of select embodiments of the present disclosure and are not meant to limit the scope of the present disclosure.

### Preparation of Complexes of the Present Disclosure

### Example 1. Preparation of 3-hydroxy-3-methylbutyric acid·sodium 3-hydroxy-3-methylbutyrate Complex

118 g of 3-hydroxy-3-methylbutyric acid, 140 g of sodium 3-hydroxy-3-methylbutyrate, 500 mL dichloromethane were added into a 1-L reaction bottle, heated to 40° C. It was stirred and dissolved, cooled to 0-10° C.; the solid was precipitated, filtered, and dried at 55° C., to obtain 180 g of 3-hydroxy-3-methylbutyric acid·sodium 3-hydroxy-3-methylbutyrate complex.

### Example 2. Preparation of 3-hydroxy-3-methylbutyric acid·sodium 3-hydroxy-3-methylbutyrate Complex

118 g of 3-hydroxy-3-methylbutyric acid, 140 g of sodium 3-hydroxy-3-methylbutyrate, 500 mL acetone were added into a 1-L reaction bottle, heated to 60° C. It was stirred and dissolved, cooled to 0-10° C.; the solid was precipitated, filtered, and dried at 55° C., to obtain 150 g of 3-hydroxy-3-methylbutyric acid·sodium 3-hydroxy-3-methylbutyrate complex.

### Example 3. Preparation of 3-hydroxy-3-methylbutyric acid·sodium 3-hydroxy-3-methylbutyrate Complex

400 mL water and 34 g sodium hydroxide were added into a 1-L reaction bottle. It was stirred and dissolved, cooled to below 25° C., and then 200 g of 3-hydroxy-3-methylbutyric acid was added. It was stirred for half an hour, and distilled under reduced pressure to remove water. After evaporation to near dryness, 400 mL dichloromethane was added. It was stirred, cooled to 0-10° C.; the solid was precipitated, filtered, and dried at 55° C., to obtain 150 g of 3-hydroxy-3-methylbutyric acid·sodium 3-hydroxy-3-methylbutyrate complex.

### Example 4. Preparation of 3-hydroxy-3-methylbutyric acid·sodium 3-hydroxy-3-methylbutyrate Complex

400 mL water and 34 g sodium hydroxide were added into a 1-L reaction bottle. It was stirred and dissolved, cooled to below 25° C., and then 200 g of 3-hydroxy-3-methylbutyric acid was added. It was stirred for half an hour, and distilled under reduced pressure to remove water. After evaporation to near dryness, 400 mL acetone was added. It was stirred, cooled to 0-10° C.; the solid was precipitated, filtered, and dried at 55° C., to obtain 110 g of 3-hydroxy-3-methylbutyric acid·sodium 3-hydroxy-3-methylbutyrate complex.

### Example 5. Preparation of 3-hydroxy-3-methylbutyric acid·sodium 3-hydroxy-3-methylbutyrate Complex

132 g of methyl 3-hydroxy-3-methylbutyrate, 360 mL water and 12 g of catalyst were added into a 1-L reaction bottle, heated at 90-95° C. for 24 hours until the reaction was complete. It was cooled to room temperature, and the catalyst was filtered off. 50 mL aqueous solution of 20 g of sodium hydroxide was added to the filtrate, and the water was distilled off under reduced pressure. After evaporation to near dryness, 100 mL dichloromethane was added. It was stirred, cooled to 0-10° C.; the solid was precipitated, filtered, and dried at 55° C., to obtain 90 g of 3-hydroxy-3-methylbutyric acid·sodium 3-hydroxy-3-methylbutyrate complex.

### Example 6. Preparation of 3-hydroxy-3-methylbutyric acid·sodium 3-hydroxy-3-methylbutyrate Complex

132 g of methyl 3-hydroxy-3-methylbutyrate, 360 mL water and 12 g of catalyst were added into a 1-L reaction bottle, heated at 90-95° C. for 24 hours until the reaction was complete. It was cooled to room temperature, and the catalyst was filtered off. 50 mL aqueous solution of 9 g of sodium hydroxide was added to the filtrate, and the water was distilled off under reduced pressure. After evaporation to near dryness, 100 mL acetone was added. It was stirred, cooled to 0-10° C.; the solid was precipitated, filtered, and dried at 55° C., to obtain 80 g of 3-hydroxy-3-methylbutyric acid·sodium 3-hydroxy-3-methylbutyrate complex.

### Example 7. Preparation of 3-hydroxy-3-methylbutyric acid·potassium 3-hydroxy-3-methylbutyrate Complex

118 g of 3-hydroxy-3-methylbutyric acid, 156 g of potassium 3-hydroxy-3-methylbutyrate, 500 mL dichloromethane were added into a 1-L reaction bottle, heated to 40° C. It was stirred and dissolved, cooled to 0-10° C.; the solid was precipitated, filtered, and dried at 55° C., to obtain 180 g of 3-hydroxy-3-methylbutyric acid·potassium 3-hydroxy-3-methylbutyrate complex.

### Example 8. Preparation of 3-hydroxy-3-methylbutyric acid·potassium 3-hydroxy-3-methylbutyrate Complex

118 g of 3-hydroxy-3-methylbutyric acid, 156 g of potassium 3-hydroxy-3-methylbutyrate, 500 mL acetone were added into a 1-L reaction bottle, heated to 60° C. It was stirred and dissolved, cooled to 0-10° C.; the solid was precipitated, filtered, and dried at 55° C., to obtain 150 g of 3-hydroxy-3-methylbutyric acid·potassium 3-hydroxy-3-methylbutyrate complex.

### Example 9. Preparation of 3-hydroxy-3-methylbutyric acid·potassium 3-hydroxy-3-methylbutyrate Complex

400 mL water and 112 g potassium hydroxide were added into a 1-L reaction bottle. It was stirred and dissolved, cooled to below 25° C., and then 200 g of 3-hydroxy-3-methylbutyric acid was added. It was stirred for half an hour, and distilled under reduced pressure to remove water. After evaporation to near dryness, 400 mL dichloromethane was added. It was stirred, cooled to 0-10° C.; the solid was precipitated, filtered, and dried at 55° C., to obtain 150 g of 3-hydroxy-3-methylbutyric acid·potassium 3-hydroxy-3-methylbutyrate complex.

### Example 10. Preparation of 3-hydroxy-3-methylbutyric acid·potassium 3-hydroxy-3-methylbutyrate Complex

400 mL water and 112 g potassium hydroxide were added into a 1-L reaction bottle. It was stirred and dissolved, cooled to below 25° C., and then 200 g of 3-hydroxy-3-methylbutyric acid was added. It was stirred for half an hour, and distilled under reduced pressure to remove water. After evaporation to near dryness, 400 mL acetone was added. It was stirred, cooled to 0-10° C.; the solid was precipitated, filtered, and dried at 55° C., to obtain 110 g of 3-hydroxy-3-methylbutyric acid·potassium 3-hydroxy-3-methylbutyrate complex.

### Example 11. Preparation of 3-hydroxy-3-methylbutyric acid·potassium 3-hydroxy-3-methylbutyrate Complex

132 g of methyl 3-hydroxy-3-methylbutyrate, 360 mL water and 12 g of catalyst were added into a 1-L reaction bottle, heated at 90-95° C. for 24 hours until the reaction was complete. It was cooled to room temperature, and the catalyst was filtered off. 50 mL aqueous solution of 33 g of potassium hydroxide was added to the filtrate, and the water was distilled off under reduced pressure. After evaporation to near dryness, 100 mL dichloromethane was added. It was stirred, cooled to 0-10° C.; the solid was precipitated, filtered, and dried at 55° C., to obtain 90 g of 3-hydroxy-3-methylbutyric acid·potassium 3-hydroxy-3-methylbutyrate complex.

### Example 12. Preparation of 3-hydroxy-3-methylbutyric acid·potassium 3-hydroxy-3-methylbutyrate Complex

132 g of methyl 3-hydroxy-3-methylbutyrate, 360 mL water and 12 g of catalyst were added into a 1-L reaction bottle, heated at 90-95° C. for 24 hours until the reaction was complete. It was cooled to room temperature, and the catalyst was filtered off. 50 mL aqueous solution of 33 g of potassium hydroxide was added to the filtrate, and the water was distilled off under reduced pressure. After evaporation to near dryness, 100 mL acetone was added. It was stirred, cooled to 0-10° C.; the solid was precipitated, filtered, and dried at 55° C., to obtain 80 g of 3-hydroxy-3-methylbutyric acid·potassium 3-hydroxy-3-methylbutyrate complex.

The 3-hydroxy-3-methylbutyric acid·calcium 3-hydroxy-3-methylbutyrate complex, 3-hydroxy-3-methylbutyric acid·magnesium 3-hydroxy-3-methylbutyrate complex and other 3-hydroxy-3-methylbutyric acid·3-hydroxy-3-methylbutyrate salt complex of the present disclosure are prepared by the same or similar method as 3-hydroxy-3-methylbutyric acid·sodium 3-hydroxy-3-methylbutyrate complex and 3-hydroxy-3-methylbutyric acid·potassium 3-hydroxy-3-methylbutyrate complex.

### Characterization of Complexes of the Present Disclosure

X-ray powder diffraction (XRPD), elemental analysis, Raman spectroscopy, infrared spectroscopy (IR), TGA, NMR, IPC-MS, DSC, DVS, etc. were performed on the complexes prepared in the Examples.

### Example 13. X-Ray Diffraction

X-ray powder diffraction patterns were obtained by using SmartLab 3KW X-ray powder diffractometer under the following conditions: diffraction line: Cu_K-beta (40 KV, 40 mA), scanning rate: 20.00 deg/min, and scanning range: 5°˜40°.

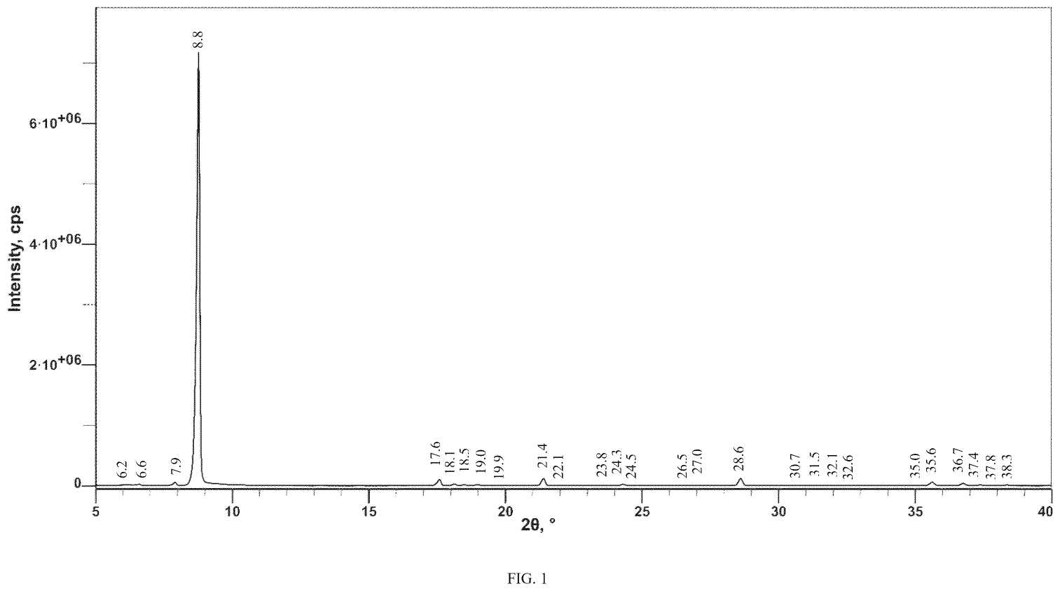

The XRPD pattern of the 3-hydroxy-3-methylbutyric acid·sodium 3-hydroxy-3-methylbutyrate complex obtained in Example 1 is shown in FIG. 1, and the XRPD data of the complex obtained in Example 1 are shown in Table 1.

TABLE 1

| position (2θ, °) | spacing (d, Å) | relative intensity (%) |
|---|---|---|
| 6.2 | 14.3 | 0.29 |
| 6.6 | 13.4 | 0.22 |
| 7.9 | 11.2 | 0.56 |
| 8.8 | 10.1 | 100 |
| 17.6 | 5.0 | 1.86 |
| 18.1 | 4.9 | 0.46 |
| 18.5 | 4.8 | 0.29 |
| 19.0 | 4.7 | 0.4 |
| 19.9 | 4.4 | 0.1 |
| 21.4 | 4.2 | 2.04 |
| 22.1 | 4.0 | 0.14 |
| 24.3 | 3.7 | 0.42 |
| 24.5 | 3.6 | 0.14 |
| 26.5 | 3.4 | 0.16 |
| 28.6 | 3.1 | 2.17 |
| 30.7 | 2.9 | 0.13 |
| 35.6 | 2.5 | 1.15 |
| 36.7 | 2.4 | 0.76 |
| 37.4 | 2.4 | 0.29 |
| 38.3 | 2.3 | 0.23 |

The XRPD results of the complexes prepared in Examples 2-6 are substantially the same as those in Example 1.

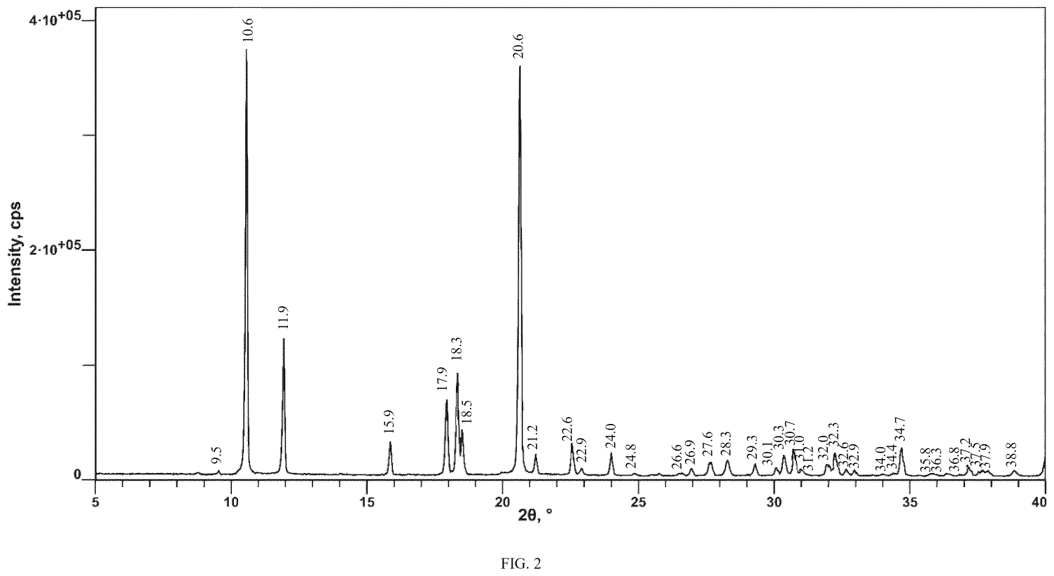

The XRPD pattern of the 3-hydroxy-3-methylbutyric acid·potassium 3-hydroxy-3-methylbutyrate complex obtained in Example 7 is shown in FIG. 2, and the XRPD data of the complex obtained in Example 7 are shown in Table 2.

TABLE 2

| position (2θ, °) | spacing (d, Å) | relative intensity (%) |
|---|---|---|
| 10.6 | 8.4 | 80.95 |
| 11.9 | 7.4 | 27.2 |
| 15.9 | 5.6 | 6.98 |
| 17.9 | 4.9 | 17.91 |
| 18.3 | 4.8 | 25.44 |
| 18.5 | 4.8 | 9.18 |
| 20.6 | 4.3 | 100 |
| 21.2 | 4.2 | 5.41 |
| 22.6 | 3.9 | 8.46 |
| 24.0 | 3.7 | 5.09 |
| 27.6 | 3.2 | 4.69 |
| 28.3 | 3.2 | 5.29 |
| 29.3 | 3.0 | 3.19 |
| 30.1 | 3.0 | 2.05 |
| 30.3 | 2.9 | 6.45 |
| 30.7 | 2.9 | 7.61 |
| 31.0 | 2.9 | 2.2 |
| 32.0 | 2.8 | 4.93 |
| 32.3 | 2.8 | 6.82 |
| 34.7 | 2.6 | 8.59 |

The XRPD results of the complexes prepared in Examples 8-12 are substantially the same as those in Example 7.

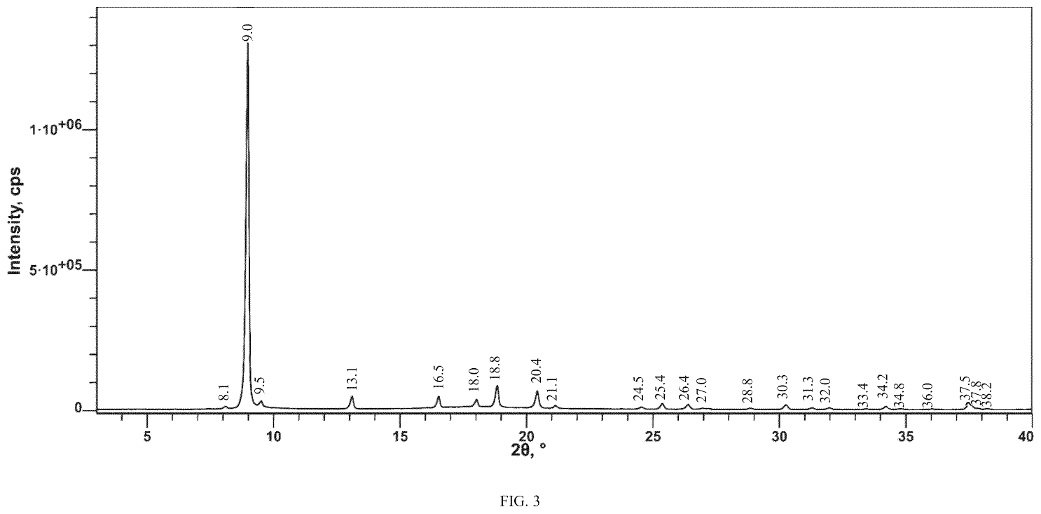

The XRPD pattern of the 3-hydroxy-3-methylbutyric acid·calcium 3-hydroxy-3-methylbutyrate complex of the present disclosure is shown in FIG. 3, and the XRPD data are shown in Table 3.

TABLE 3

| position (2θ, °) | spacing (d, Å) | relative intensity (%) |
|---|---|---|
| 8.1 | 10.9 | 0.43 |
| 9.0 | 9.8 | 100 |
| 9.5 | 9.3 | 3.13 |
| 13.1 | 6.8 | 3.89 |
| 16.5 | 5.4 | 2.89 |
| 18.0 | 4.9 | 2.59 |
| 18.8 | 4.7 | 6.71 |
| 20.4 | 4.3 | 5.36 |
| 21.1 | 4.2 | 0.72 |
| 24.5 | 3.6 | 0.77 |
| 25.4 | 3.5 | 1.76 |
| 26.4 | 3.4 | 1.54 |
| 27.0 | 3.3 | 0.69 |
| 30.3 | 3.0 | 1.49 |
| 31.3 | 2.9 | 0.6 |
| 32.0 | 2.8 | 0.62 |
| 34.2 | 2.6 | 1.23 |
| 36.0 | 2.5 | 0.33 |
| 37.5 | 2.4 | 2.8 |
| 38.2 | 2.4 | 0.4 |

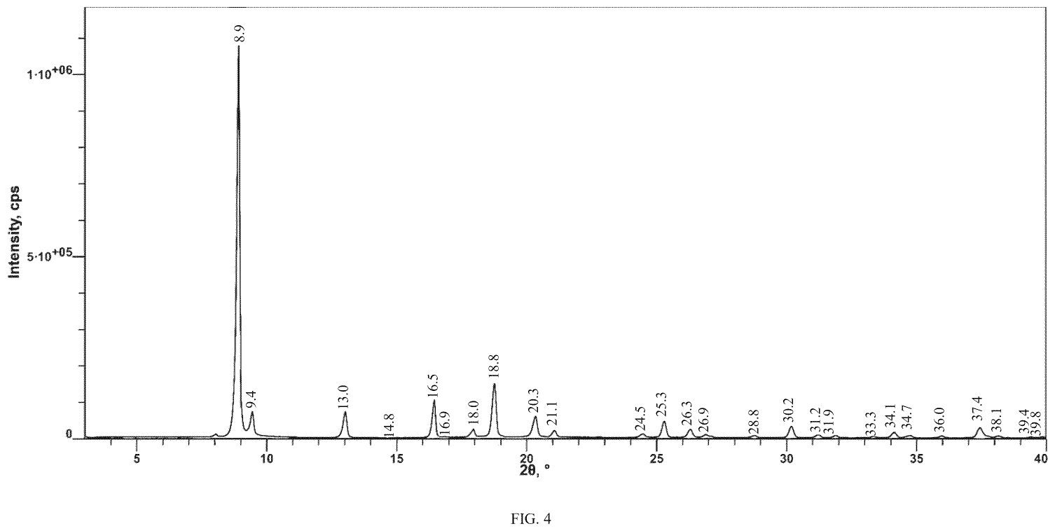

The XRPD pattern of the 3-hydroxy-3-methylbutyric acid·magnesium 3-hydroxy-3-methylbutyrate complex of the present disclosure is shown in FIG. 4, and the XRPD data are shown in Table 4.

TABLE 4

| position (2θ, °) | spacing (d, Å) | relative intensity (%) |
|---|---|---|
| 8.9 | 9.9 | 100 |
| 9.4 | 9.4 | 8.66 |
| 13.0 | 6.8 | 7.86 |
| 16.5 | 5.4 | 10.86 |
| 18.0 | 4.9 | 2.43 |
| 18.8 | 4.7 | 17.36 |
| 20.3 | 4.4 | 7.38 |
| 21.1 | 4.2 | 1.86 |
| 24.5 | 3.6 | 1.49 |
| 25.3 | 3.5 | 5.34 |
| 26.3 | 3.4 | 2.93 |
| 26.9 | 3.3 | 1.44 |
| 28.8 | 3.1 | 0.75 |
| 30.2 | 3.0 | 3.84 |
| 31.2 | 2.9 | 1.01 |
| 31.9 | 2.8 | 0.73 |
| 33.3 | 2.7 | 0.51 |
| 34.1 | 2.6 | 2.04 |
| 36.0 | 2.5 | 0.64 |
| 37.4 | 2.4 | 5.18 |

### Example 14. Infrared Spectroscopy (IR)

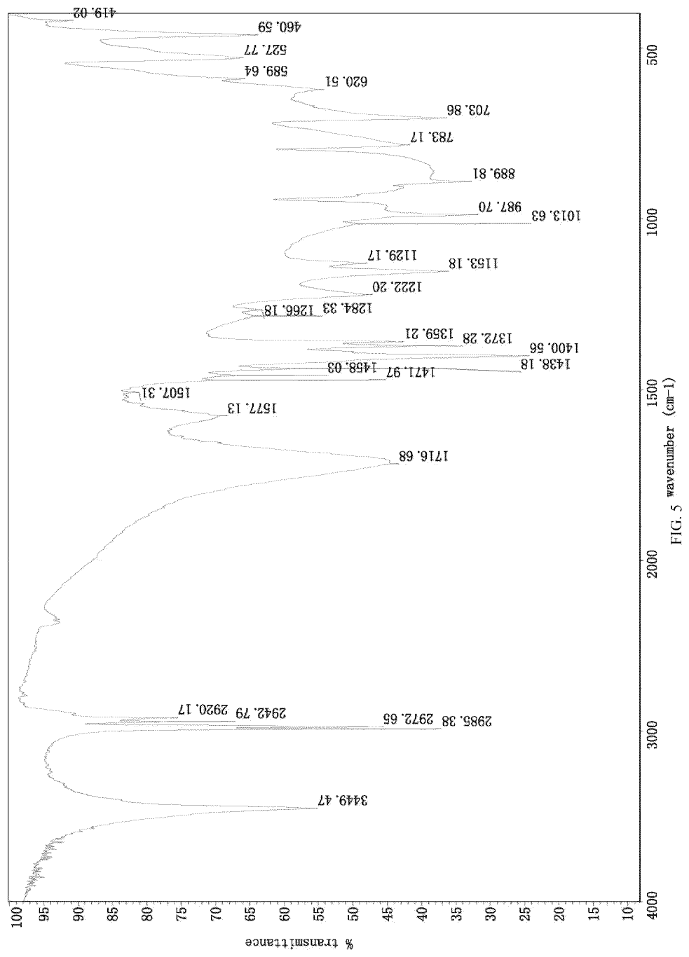

The infrared spectrum of the complex of Example 1 was analyzed by Fourier transform attenuated total reflection infrared spectrometer. FIG. 5 shows an infrared spectrogram (IR) of the complex of Example 1. As shown in FIG. 5, the complex has characteristic absorption peaks at 3449 cm⁻¹, 2972 cm⁻¹, 2942 cm⁻¹, 2920 cm⁻¹, 1713 cm⁻¹, 1577 cm⁻¹, 1507 cm⁻¹, 1459 cm⁻¹, 1438 cm⁻¹, 1401 cm⁻¹, 1372 cm⁻¹, 1286 cm⁻¹, 1255 cm⁻¹, 1220 cm⁻¹, 1152 cm⁻¹, 987 cm⁻¹, 913 cm⁻¹, 891 cm⁻¹, 860 cm⁻¹, 782 cm⁻¹, 705 cm⁻¹, 619 cm⁻¹, 529 cm⁻¹, 502 cm⁻¹, 462 cm⁻¹, 413 cm⁻¹. The IR results of the complexes prepared in Examples 2-6 are substantially the same as those in Example 1.

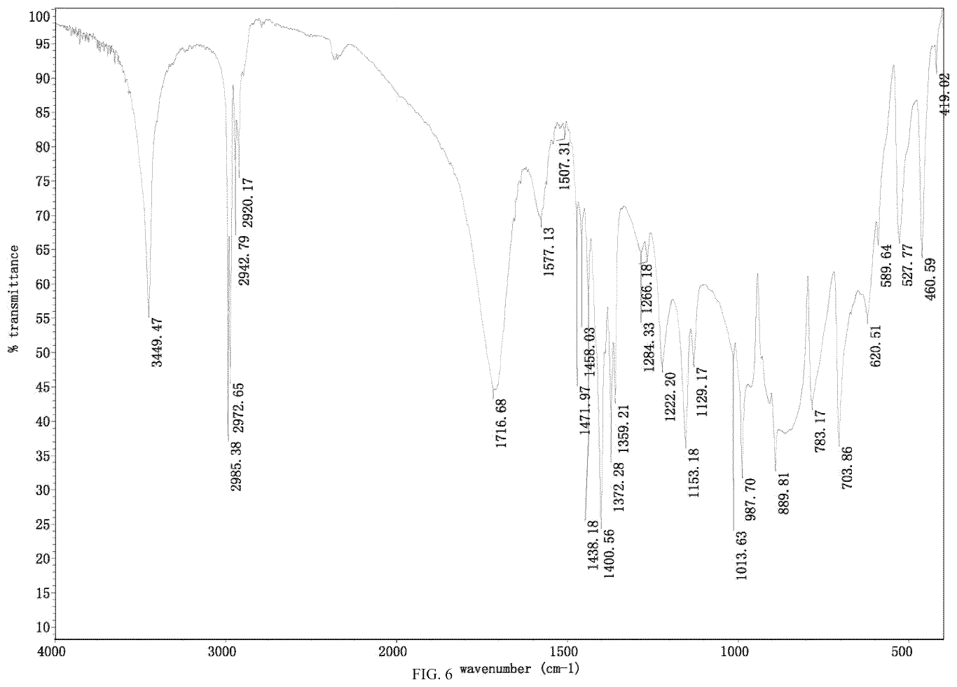

The infrared spectrum of the complex of Example 7 was analyzed by Fourier transform attenuated total reflection infrared spectrometer. FIG. 6 shows an infrared spectrogram (IR) of the complex of Example 7. As shown in FIG. 6, the complex has characteristic absorption peaks at 3449 cm⁻¹, 2985 cm⁻¹, 2973 cm⁻¹, 2943 cm⁻¹, 2920 cm⁻¹, 1717 cm⁻¹, 1577 cm⁻¹, 1507 cm⁻¹, 1472 cm⁻¹, 1458 cm⁻¹, 1438 cm⁻¹, 1401 cm⁻¹, 1372 cm⁻¹, 1359 cm⁻¹, 1284 cm⁻¹, 1266 cm⁻¹, 1222 cm⁻¹, 1153 cm⁻¹, 1129 cm⁻¹, 1014 cm⁻¹, 988 cm⁻¹, 890 cm⁻¹, 783 cm⁻¹, 704 cm⁻¹, 621 cm⁻¹, 590 cm⁻¹, 528 cm⁻¹, 461 cm⁻¹, 419 cm⁻¹. The IR results of the complexes prepared in Examples 8-12 are substantially the same as those in Example 7.

The infrared spectrum of the 3-hydroxy-3-methylbutyric acid·calcium 3-hydroxy-3-methylbutyrate complex of the present disclosure was analyzed. FIG. 7 shows an infrared spectrogram (IR) thereof. As shown, the complex has characteristic absorption peaks at 2970 cm⁻¹, 1713 cm⁻¹, 1591 cm⁻¹, 1476 cm⁻¹, 1412 cm⁻¹, 1369 cm 1, 1200 cm⁻¹, 1132 cm⁻¹, 989 cm⁻¹, 959 cm⁻¹, 920 cm⁻¹, 897 cm⁻¹, 818 cm⁻¹, 793 cm⁻¹, 768 cm⁻¹, 750 cm⁻¹, 710 cm⁻¹, 633 cm⁻¹, 546 cm⁻¹, 467 cm⁻¹, 430 cm⁻¹.

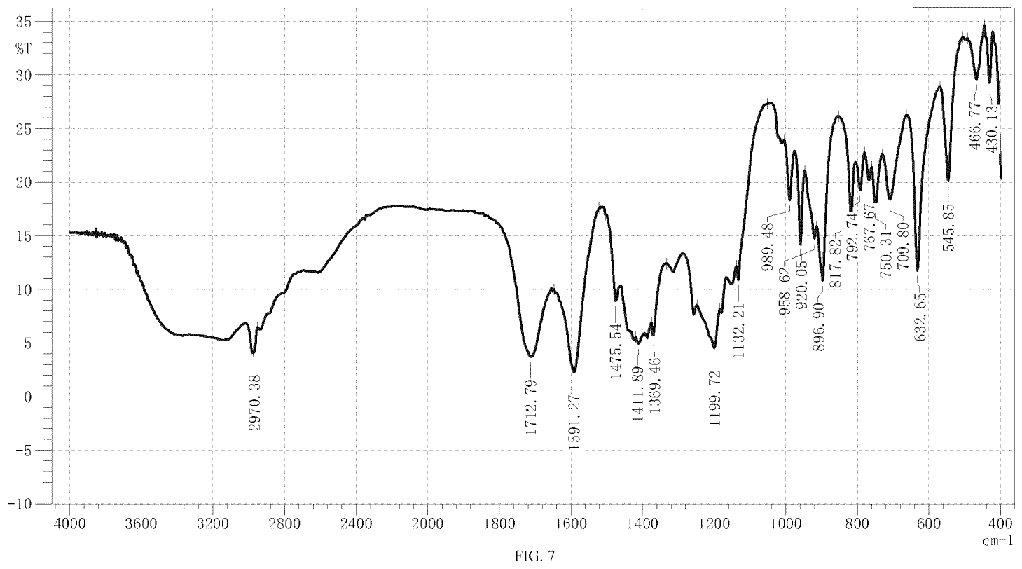

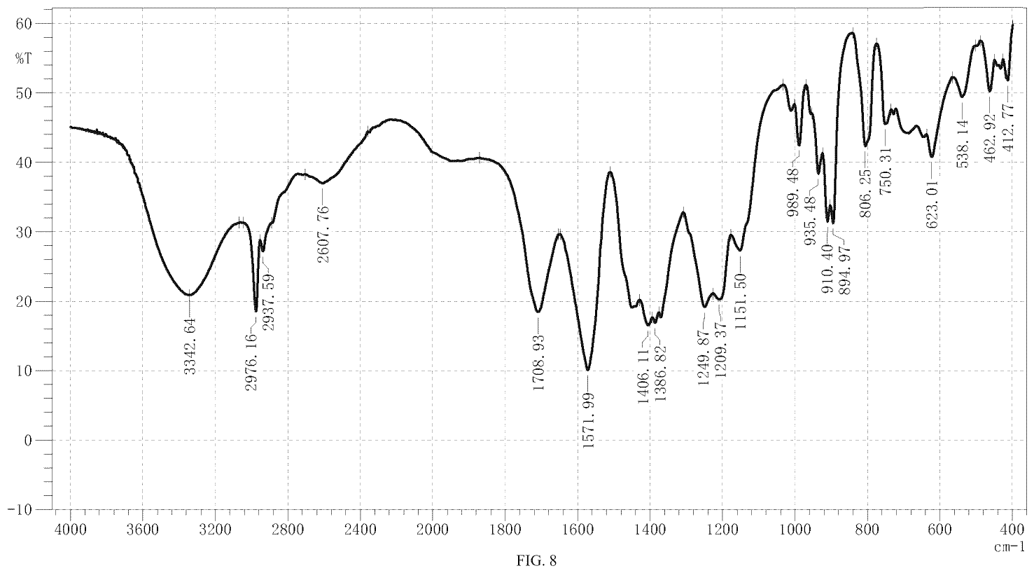

The infrared spectrum of the 3-hydroxy-3-methylbutyric acid·magnesium 3-hydroxy-3-methylbutyrate complex of the present disclosure was analyzed. FIG. 8 shows an infrared spectrogram (IR) thereof. As shown, the complex has characteristic absorption peaks at 3343 cm⁻¹, 2976 cm⁻¹, 2938 cm⁻¹, 2608 cm⁻¹, 1709 cm⁻¹, 1572 cm 1, 1406 cm⁻¹, 1387 cm⁻¹, 1250 cm⁻¹, 1209 cm⁻¹, 1152 cm⁻¹, 989 cm⁻¹, 935 cm⁻¹, 910 cm 1, 895 cm⁻¹, 806 cm⁻¹, 750 cm⁻¹, 623 cm⁻¹, 538 cm⁻¹, 463 cm⁻¹, 413 cm⁻¹.

### Example 15. Thermogravimetric Analysis (TGA)

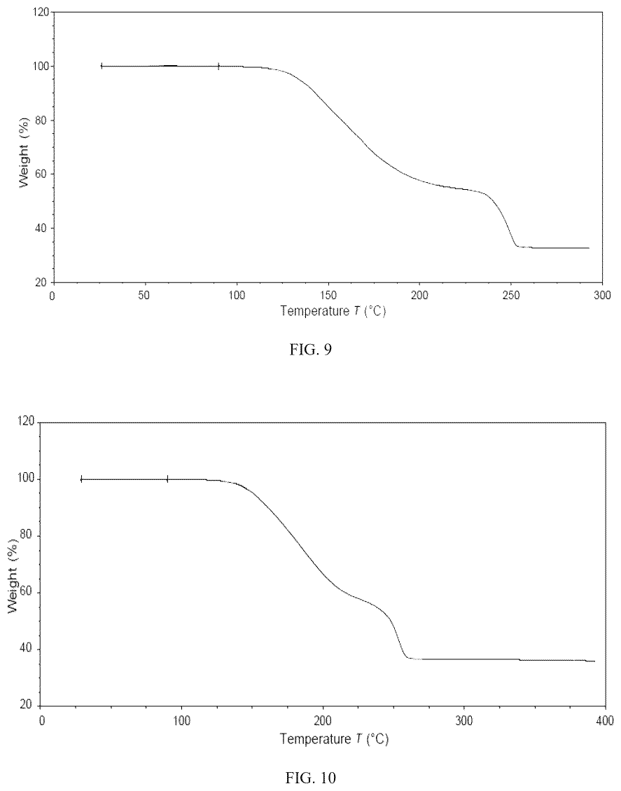

Thermogravimetric analysis (TGA) was carried out on the complex of Example 1, and the TGA curve is shown in FIG. 9, which depicts the percentage of weight loss of the sample as a function of temperature. The TGA results of the complexes prepared in Examples 2-6 are substantially the same as those in Example 1. Thermogravimetric analysis (TGA) was carried out on the complex of Example 7, and the TGA curve is shown in FIG. 10, which depicts the percentage of weight loss of the sample as a function of temperature. The TGA results of the complexes prepared in Examples 8-12 are substantially the same as those in Example 7.

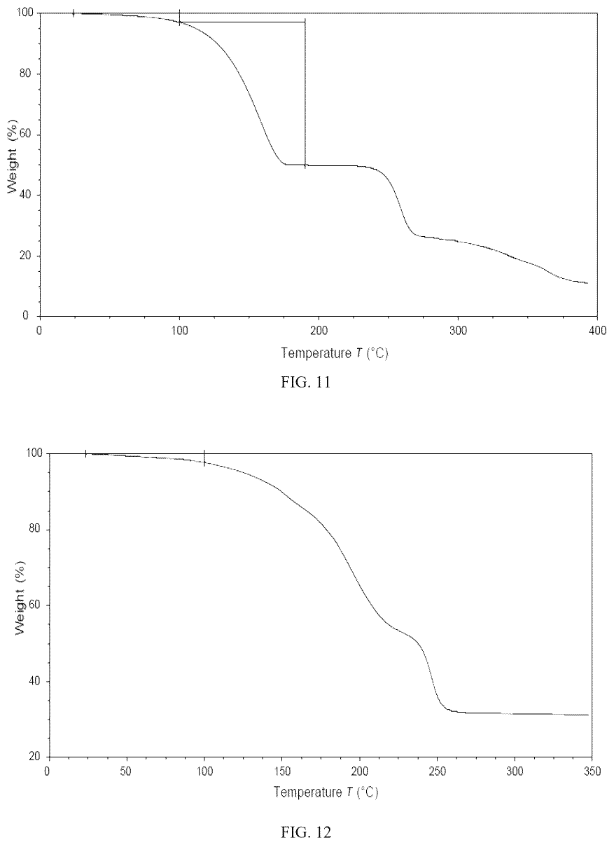

Thermogravimetric analysis was carried out on the 3-hydroxy-3-methylbutyric acid·calcium 3-hydroxy-3-methylbutyrate complex of the present disclosure, and the TGA curve is shown in FIG. 11, it has a weight loss of 2.98% when heated from 24.2° C. to 100.0° C. Thermogravimetric analysis was carried out on the 3-hydroxy-3-methylbutyric acid·magnesium 3-hydroxy-3-methylbutyrate complex of the present disclosure, and the TGA curve is shown in FIG. 12, it has a weight loss of 2.30% when heated from 23.5° C. to 100.0° C.

### Example 16. Elemental Analysis

Elemental analysis was performed on the complex of Example 1 by elemental analyzer: C, 45.34%; H, 7.13%. The elemental analysis results of the complexes prepared in Examples 2-6 are substantially the same as those in Example 1. Elemental analysis was performed on the complex of Example 7 by elemental analyzer: C, 46.73%; H, 7.43%. The elemental analysis results of the complexes prepared in Examples 8-12 are substantially the same as those in Example 7.

### Example 17. Raman Spectroscopy

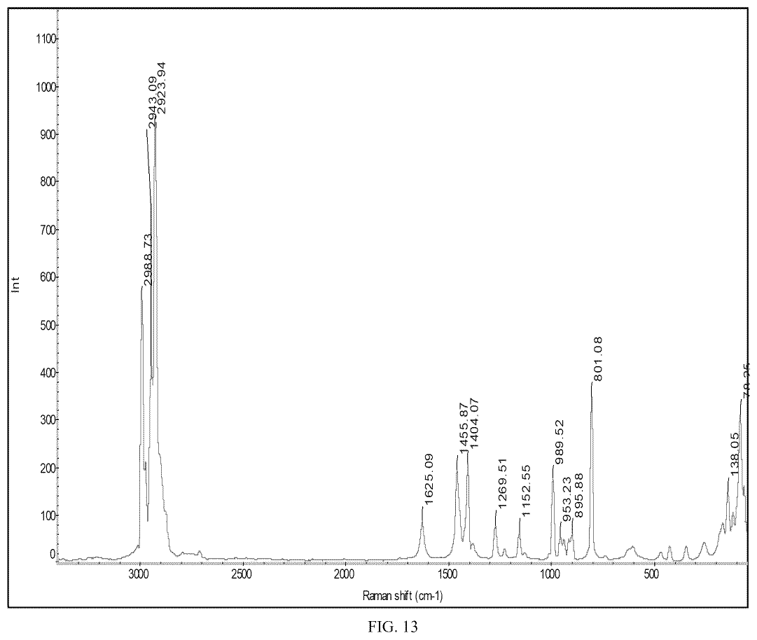

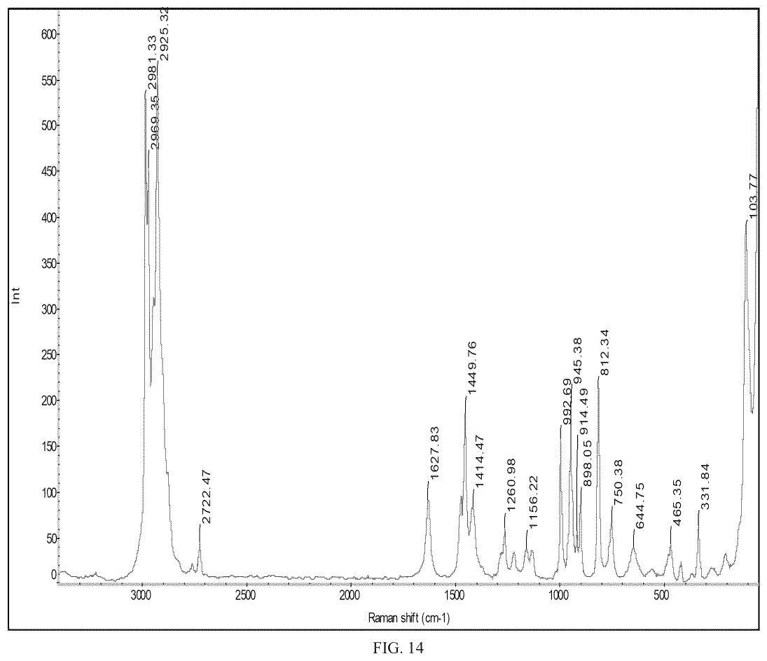

The complex of Example 1 was analyzed by Raman spectroscopy, and the results are shown in FIG. 13. The Raman spectra of the complexes prepared in Examples 2-6 are substantially the same as those in Example 1. The complex of Example 7 was analyzed by Raman spectroscopy, and the results are shown in FIG. 14. The Raman spectra of the complexes prepared in Examples 8-12 are substantially the same as those in Example 7.

### Example 18. NMR Determination

¹H NMR spectrum of the complex of Example 1 was recorded in a spectrometer: ¹H NMR (400 MHz, D₂O) δ 2.36 (s, 2H), 1.20 (s, 6H). The NMR results of the complexes prepared in Examples 2-6 are substantially the same as those in Example 1. ¹H NMR spectrum of the complex of Example 7 was recorded in a spectrometer: ¹H NMR (400 MHz, D₂O) δ 2.36 (s, 2H), 1.20 (s, 6H). The NMR results of the complexes prepared in Examples 8-12 are substantially the same as those in Example 7. The NMR results of the 3-hydroxy-3-methylbutyric acid calcium 3-hydroxy-3-methylbutyrate complex and 3-hydroxy-3-methylbutyric acid·magnesium 3-hydroxy-3-methylbutyrate complex of the present disclosure are both substantially the same as those in Examples 1 and 7.

### Example 19. Differential Scanning Calorimetry (DSC)

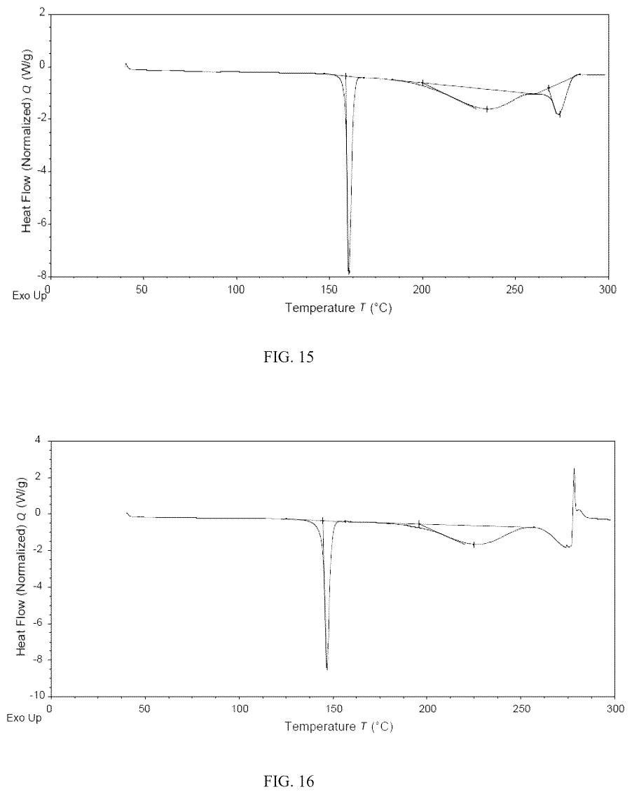

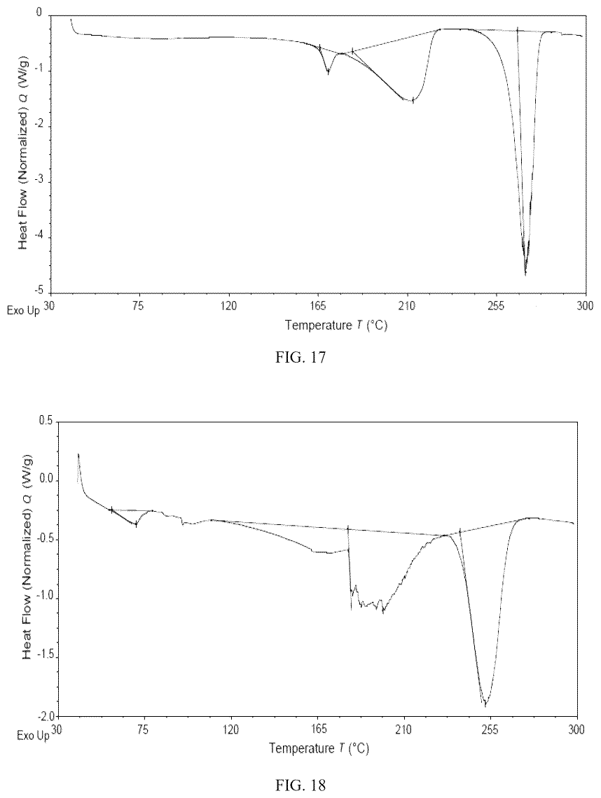

FIG. 15 shows a DSC thermogram of the 3-hydroxy-3-methylbutyric acid·sodium 3-hydroxy-3-methylbutyrate complex of the present disclosure, which contains an endothermic peak of 160.28° C.±3° C. FIG. 16 shows a DSC thermogram of the 3-hydroxy-3-methylbutyric acid·potassium 3-hydroxy-3-methylbutyrate complex of the present disclosure, which contains an endothermic peak of 146.73° C.±3° C. FIG. 17 shows a DSC thermogram of the 3-hydroxy-3-methylbutyric acid·calcium 3-hydroxy-3-methylbutyrate complex of the present disclosure, which contains an endothermic peak of 269.38° C.±3° C. FIG. 18 shows a DSC thermogram of the 3-hydroxy-3-methylbutyric acid·magnesium 3-hydroxy-3-methylbutyrate complex of the present disclosure, which contains an endothermic peak of 252.26° C.±3° C.

### Example 20

The 3-hydroxy-3-methylbutyric acid content and metal ion content were measured for the complexes of each Example, and the results are shown in Table 5.

TABLE 5

| | 3-hydroxy-3- | | | |
|---|---|---|---|---|
| | methylbutyric acid | | Metal ion | |
| | Theoretical | Measured | Theoretical | Measured |
| Sample | content | content | content | content |
| HMB-Na | 91.09% | 90.61% | 8.91% | 9.39% |
| complex | | | | |
| HMB-K | 85.75% | 85.50% | 14.25% | 14.50% |
| complex | | | | |
| HMB-Ca | 92.15% | 92.08% | 7.85% | 7.92% |
| complex | | | | |
| HMB-Mg | 95.09% | 95.05% | 4.91% | 4.95% |
| complex | | | | |

### Example 21

The solubility of various 3-hydroxy-3-methylbutyrate salts and the complexes of Examples was each determined. As shown in Table 6, the solubility of the complex of the present disclosure was each significantly higher than that of the corresponding 3-hydroxy-3-methylbutyrate salt.

TABLE 6

| Sample | Solubility (g/100 |
|---|---|
| sodium 3-hydroxy-3- | 72.9 |
| HMB-Na complex | 135 |
| potassium 3-hydroxy-3- | 101.1 |
| HMB-K complex | 225 |
| calcium 3-hydroxy-3- | 15 |
| HMB-Ca complex | 32 |
| magnesium 3-hydroxy-3- | 6 |
| HMB-Mg complex | 13 |

### Example 22

The stability of various 3-hydroxy-3-methylbutyrate salts and the complexes of the present disclosure was each measured at 23° C. and 92.5% humidity, and the results are shown in Table 7.

TABLE 7

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | sodium 3-hydroxy-3- | 0.32 | 0.65 | 1.07 | 1.98 | 2.32 | 2.58 | 3.13 | 5.47 |
| 23° C. | methylbutyrate | | | | | | | | |
| humidity | HMB-Na complex | 0.22 | 0.46 | 0.75 | 1.39 | 1.62 | 1.81 | 2.19 | 3.83 |
| 92.5% | potassium 3- | 0.42 | 0.85 | 1.39 | 2.57 | 3.02 | 3.35 | 4.07 | 7.11 |
| | hydroxy-3- | | | | | | | | |
| | methylbutyrate | | | | | | | | |
| | HMB-K complex | 0.29 | 0.59 | 0.97 | 1.80 | 2.11 | 2.35 | 2.85 | 4.98 |
| | calcium 3-hydroxy- | 0.19 | 0.38 | 0.63 | 1.16 | 1.36 | 1.51 | 1.83 | 3.20 |
| | 3-methylbutyrate | | | | | | | | |
| | HMB-Ca complex | 0.14 | 0.29 | 0.47 | 0.87 | 1.02 | 1.13 | 1.37 | 2.40 |
| | magnesium 3- | 0.11 | 0.23 | 0.38 | 0.70 | 0.81 | 0.91 | 1.10 | 1.92 |
| | hydroxy-3- | | | | | | | | |
| | methylbutyrate | | | | | | | | |
| | HMB-Mg complex | 0.08 | 0.17 | 0.28 | 0.52 | 0.61 | 0.68 | 0.82 | 1.44 |

### Example 23

Thirty-two 24-month-old female C57BL/6 mice were randomly divided into four groups, with 8 mice in each group. The four groups were given water (NC group); commercially available calcium 3-hydroxy-3-methylbutyrate (HMB calcium salt); the HMB-Na complex of the present disclosure; and the HMB-Ca compound of the present disclosure by gavage respectively, once a day for about 6 weeks. The final concentration of HMB was about 384 mg/kg. At the seventh week, the hind limb muscles were dissected and weighed, and the data were recorded.

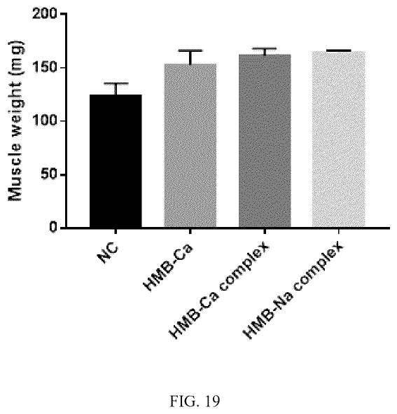

FIG. 19 shows the muscle weight results of experimental mice at 7 weeks. As shown in FIG. 19, compared with the existing HMB calcium salt products, the muscle weight of mice administered with the complex of the present disclosure has obviously increased.

The complex of the present disclosure has high HMB content, high solubility and good stability, effectively solves the problems of low solubility, poor stability, acid/salt load, excessive calcium intake and gastrointestinal side effects associated with existing products, has good taste comfort, and improves flavor. In addition, compared with the existing HMB calcium salt, the complex of the present disclosure has the advantages of better increasing muscle weight, improving muscle performance, joint function, body composition or enhancing health due to changes in solubility and stability. It can be better used as a dietary or nutritional supplement.

The above are only the preferred embodiments of the present disclosure, and are not intended to limit the present disclosure. Those skilled in the art can make many changes, modifications, substitutions and variations on these embodiments without departing from the principles and purposes of the present disclosure, and the scope of the present disclosure is defined by the claims and their equivalents.

What is claimed is:

1. A 3-hydroxy-3-methylbutyric acid·3-hydroxy-3-methylbutyrate complex and composition thereof, wherein a salt in the 3-hydroxy-3-methylbutyric acid·3-hydroxy-3-methylbutyrate complex is a sodium salt, potassium salt, calcium salt, magnesium salt, ammonium salt, amino acid salt.

2. The 3-hydroxy-3-methylbutyric acid·3-hydroxy-3-methylbutyrate complex according to claim 1, wherein the 3-hydroxy-3-methylbutyric acid·3-hydroxy-3-methylbutyrate complex is in a solid form.

3. The 3-hydroxy-3-methylbutyric acid·3-hydroxy-3-methylbutyrate complex according to claim 1, wherein the 3-hydroxy-3-methylbutyric acid·3-hydroxy-3-methylbutyrate complex is in crystalline form.

4. The 3-hydroxy-3-methylbutyric acid·3-hydroxy-3-methylbutyrate complex according to claim 1, wherein the complex has the following structure:

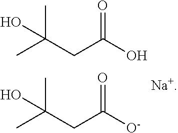

5. The 3-hydroxy-3-methylbutyric acid·3-hydroxy-3-methylbutyrate complex according to claim 4, wherein an X-ray powder diffraction pattern of the complex comprises peaks at diffraction angles (2θ) of 7.9±0.2°, 8.8±0.2°, 17.6±0.2°, 18.1±0.2°, 19.0±0.2°, 21.4±0.2°, 24.3±0.2°, 28.6±0.2°, 35.6±0.2°, and 36.7±0.2°.

6. The 3-hydroxy-3-methylbutyric acid·3-hydroxy-3-methylbutyrate complex according to claim 4, wherein an X-ray powder diffraction pattern of the complex is shown in FIG. 1.

7. The 3-hydroxy-3-methylbutyric acid·3-hydroxy-3-methylbutyrate complex according to claim 1, wherein the complex has the following structure:

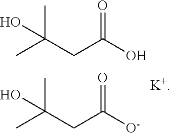

8. The 3-hydroxy-3-methylbutyric acid·3-hydroxy-3-methylbutyrate complex according to claim 7, wherein an X-ray powder diffraction pattern of the complex comprises peaks at diffraction angles (2θ) of 10.6±0.2°, 11.9±0.2°, 15.9±0.2°, 17.9±0.2°, 18.3±0.2°, 18.5±0.2°, 20.6±0.2°, 22.6±0.2°, 30.7±0.2°, and 34.7±0.2°.

9. The 3-hydroxy-3-methylbutyric acid·3-hydroxy-3-methylbutyrate complex according to claim 7, wherein an X-ray powder diffraction pattern of the complex is shown in FIG. 2.

10. The 3-hydroxy-3-methylbutyric acid·3-hydroxy-3-methylbutyrate complex according to claim 1, wherein the complex has the following structure:

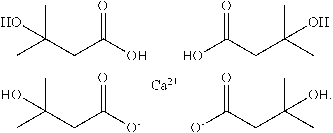

11. The 3-hydroxy-3-methylbutyric acid·3-hydroxy-3-methylbutyrate complex according to claim 10, wherein an X-ray powder diffraction pattern of the complex comprises peaks at diffraction angles (2θ) of 8.1±0.2°, 9.0±0.2°, 13.1±0.2°, 16.5±0.2°, 18.8±0.2°, 20.4±0.2°, 25.4±0.2°, 26.4±0.2°, 30.3±0.2°, and 34.2±0.2°.

12. The 3-hydroxy-3-methylbutyric acid·3-hydroxy-3-methylbutyrate complex according to claim 10, wherein an X-ray powder diffraction pattern of the complex is shown in FIG. 3.

13. The 3-hydroxy-3-methylbutyric acid·3-hydroxy-3-methylbutyrate complex according to claim 1, wherein the complex has the following structure:

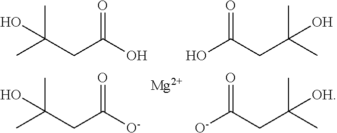

14. The 3-hydroxy-3-methylbutyric acid·3-hydroxy-3-methylbutyrate complex according to claim 13, wherein an X-ray powder diffraction pattern of the complex comprises peaks at diffraction angles (2θ) of 8.9±0.2°, 9.4±0.2°, 13.0±0.2°, 16.5±0.2°, 18.8±0.2°, 20.3±0.2°, 25.3±0.2°, 30.2±0.2°, 34.1±0.2°, and 37.4±0.2°.

15. The 3-hydroxy-3-methylbutyric acid·3-hydroxy-3-methylbutyrate complex according to claim 13, wherein an X-ray powder diffraction pattern of the complex is shown in FIG. 4.

16. The 3-hydroxy-3-methylbutyric acid·3-hydroxy-3-methylbutyrate complex according to claim 1, wherein the 3-hydroxy-3-methylbutyric acid·3-hydroxy-3-methylbutyrate complex is prepared as food, beverage, supplement, nutritional product or pharmaceutical preparation.

17. A 3-hydroxy-3-methylbutyric acid·3-hydroxy-3-methylbutyrate complex, wherein the 3-hydroxy-3-methylbutyric acid·3-hydroxy-3-methylbutyrate complex is obtained by the following method:
(1) obtaining a substance B by one of the following methods: mixing 3-hydroxy-3-methylbutyric acid and 3-hydroxy-3-methylbutyrate salt; or adding the 3-hydroxy-3-methylbutyric acid into an aqueous solution of an alkaline compound, stirring, removing water; or reacting alkyl 3-hydroxy-3-methylbutyrate with water under heating in a presence of a catalyst, cooling and filtering to yield a filtrate, adding the aqueous solution of the alkaline compound to the filtrate, distilling under reduced pressure to remove the water;
(2) adding one or more solvents selected from the following into the substance B obtained in step (1): water, tetrahydrofuran (THF), dimethylformamide (DMF), dimethyl Sulfoxide (DMSO), dimethylacetamide (DMAC), alcohol, halogenated hydrocarbon, ketone, and ester, stirring and cooling, to precipitate a solid;
(3) filtering out the solid and drying, to obtain the 3-hydroxy-3-methylbutyric acid·3-hydroxy-3-methylbutyrate complex.

18. A method for preparing the 3-hydroxy-3-methylbutyric acid·3-hydroxy-3-methylbutyrate complex according to claim 1, comprising the following steps:
(1) obtaining a substance B by one of the following methods: mixing 3-hydroxy-3-methylbutyric acid and 3-hydroxy-3-methylbutyrate salt; or adding the 3-hydroxy-3-methylbutyric acid into an aqueous solution of an alkaline compound, stirring, removing water; or reacting alkyl 3-hydroxy-3-methylbutyrate with water under heating in a presence of a catalyst, cooling and filtering to yield a filtrate, adding the aqueous solution of the alkaline compound to the filtrate, distilling under reduced pressure to remove the water;
(2) adding one or more solvents selected from the following into the substance B obtained in step (1): water, tetrahydrofuran (THF), dimethylformamide (DMF), dimethyl Sulfoxide (DMSO), dimethylacetamide (DMAC), alcohol, halogenated hydrocarbon, ketone, ester, stirring and cooling, to precipitate a solid;
(3) filtering out the solid and drying, to obtain the 3-hydroxy-3-methylbutyric acid·3-hydroxy-3-methylbutyrate complex.

19. The method according to claim 18, wherein in the step (2), the alcohol is methanol, ethanol, isopropanol, or n-butanol; the halogenated hydrocarbon is chlorobenzene, dichlorobenzene, or dichloromethane; the ketone is acetone, methyl butanone, or methyl isobutyl ketone; and the ester is ethyl acetate, isopropyl acetate, n-butyl acetate, or isobutyl acetate.

20. The method according to claim 18, wherein in the step (1), the alkaline compound is hydroxide, carbonate, bicarbonate, methoxide, acetate or formate of sodium, potassium, calcium or magnesium, ammonia water, triethylamine, N,N-diisopropylethylamine; and the alkyl 3-hydroxy-3-methylbutyrate is methyl 3-hydroxy-3-methylbutyrate, ethyl 3-hydroxy-3-methylbutyrate, propyl 3-hydroxy-3-methylbutyrate, isopropyl 3-hydroxy-3-methylbutyrate, butyl 3-hydroxy-3-methylbutyrate, or isobutyl 3-hydroxy-3-methylbutyrate.
